(19) United States
(12) Patent Application Publication
(10) Pub. No.: US 2004/0088009 A1
(43) Pub. Date: May 6, 2004

(54) Auxilary central nervous system pre-pulse for shock pain inhibition

(76) Inventor: Paul J. Degroot, Brooklyn Park, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/284,872

(22) Filed: Oct. 31, 2002

(51) Int. Cl. A61N 1/39

(52) U.S. Cl. 607/5

(57) ABSTRACT

Apparatus and an associated method are provided for detecting a cardiac arrhythmia and delivering cardioversion therapy after first delivering a prepulse inhibition stimulus directly to the central nervous system for inhibiting cardioversion pain perceived by the patient. Circuitry for controlling and delivering a prepulse stimulus may be included in the cardioverting device or in a separate stimulating device that is in communication with the cardioverting device. The prepulse stimulus is delivered directly to the spinal cord via a spinal cord lead at a predetermined time interval prior to cardioversion shock delivery.

### FIELD OF THE INVENTION

The present invention relates generally to an implantable device for delivering a pain inhibiting stimulation pulse to the central nervous system prior to delivering cardioversion shock therapy.

### BACKGROUND OF THE INVENTION

Implantable cardioverter defibrillators (ICDs) are capable of detecting cardiac arrhythmias and delivering electrical stimulation therapies to terminate arrhythmias. Tachycardia may be terminated by anti-tachycardia pacing therapies or high-voltage cardioversion shocks. Fibrillation may be terminated by high-voltage defibrillation shocks. These high-voltage shocks, which are referred to inclusively herein as “cardioversion shocks,” can be life-saving to a patient but can be very painful. Some patients have recurring arrhythmias and are subject to repeated shock therapies. Patient anxiety over receiving a painful shock therapy can affect a patient's overall quality of life and their acceptance of ICD use.

Some types of arrhythmias, such as atrial fibrillation may not be directly life-threatening but may put a patient at risk for developing more serious ventricular tachycardia or fibrillation, stroke, or injuries due to dizziness or loss of consciousness. Therefore, while not immediately life-threatening, it may be desirable to treat atrial arrhythmias with cardioversion shocks in order to prevent precipitating complications. Such treatment, however, may not be readily accepted by a patient due to the cardioversion pain to which he or she will be subjected.

One approach for reducing the pain associated with cardioversion shocks is to minimize the energy of the shocking pulse. While this approach may reduce the amount of pain perceived by the patient, it does not eliminate the pain and potentially compromises the effectiveness of the shock therapy.

Another approach to alleviating cardioversion pain is to deliver an analgesic therapy prior to delivering a cardioversion shock. An implantable cardioverter for providing cardioversion electrical energy and applying a pain alleviating therapy at an appropriate site in the patient's body prior to or in conjunction with the delivery of the cardioversion energy is generally disclosed in U.S. Pat. No. 5,662,689, issued to Elsberry et al., incorporated herein by reference in its entirety. The pain alleviating therapy for the associated cardioversion energy induced and propagated pain is preferably either an analgesic drug or electrical neurostimulation to one or more specific sites of the peripheral and central pain pathways. An analgesic drug may require a few minutes to one hour to suppress pain, depending on the specific analgesic administered. Delivery of an analgesic drug may be useful in alleviating pain associated with atrial cardioversion since rapid cardioversion is not necessary for atrial fibrillation as opposed to ventricular fibrillation.

The alleviation of pain through spinal cord stimulation (SCS) is practiced clinically and commercial devices, such as the Medtronic Itrel®II implantable neurostimulation system, are widely available for treating intractable pain. Spinal cord stimulation has also been proposed for relieving pain associated with angina as generally disclosed in U.S. Pat. No. 5,824,021, issued to Rise. See also, for example, Mannheimer C, et al., “Effects of spinal cord stimulation in angina pectoris induced by pacing and possible mechanism of action,” BMJ, 1993;307:477-80. It is postulated that spinal cord stimulation relieves pain by inhibiting impulse transmission in small fiber afferents by the activation of the large fiber afferents on the spinal segmental level. See Eliasson T, et al., “Spinal cord stimulation in angina pectoris with normal coronary arteriograms,” Coronary Artery Disease, 1993;4:819-27.

Another approach to reducing the pain that a patient experiences during cardioversion is to deliver pain-inhibiting stimuli prior to delivering the therapeutic painful stimulus as generally disclosed in U.S. Pat. No. 6,438,418, issued to Swerdlow et al., incorporated herein by reference in its entirety. Prepulse inhibition (PPI) is the suppression of a patient's perception of the intensity of and the motor response to a startling or painful stimulus by preceding the painful stimulus with a significantly less intense pre-stimulus (see, for example, Cohen et al, “Sensory magnitude estimation in the context of reflex modification,” J Exper Psychology 1981;7:1363-70, and Swerdlow et al, “Neurophysiology and neuropharmacology of short lead interval startle modification,” in Startle Modification: Implication for Neuroscience, Cognitive Science, and Clinical Science, ed. Dawson et al., Cambridge Univ. Press, 1997, Chapter 6). Prepulse inhibition is effective when a prepulse stimulus is delivered on the order of 30 to 500 ms prior to a more intense, painful stimulus.

The effectiveness of prepulse inhibition decreases when a prepulse stimulus is delivered more than one second prior to a painful stimulus. Therefore, the timing of prepulse stimuli is important in achieving a desired pain-inhibiting effect. The short time delay required between a prepulse stimulus and a painful stimulus may be used advantageously in inhibiting cardioversion shock pain since the prepulse stimulus may be delivered just prior to an urgently needed cardioversion shock. Pain inhibition may be achieved without a clinically significant delay in delivering the cardioversion shock.

The PPI effect may be realized by delivering prepulse stimuli along the same or a different sensory pathway than the painful stimulus. PPI is thought to activate sensorimotor gating processing regulated by the forebrain, thus any sensory pathway that activates this forebrain circuitry may be effective in inducing the PPI pain suppression effects. Perhaps the most direct pathway to this forebrain circuitry may be through the central nervous system itself. What is needed therefore, is a method and apparatus for reducing or eliminating cardioversion shock pain that activates the prepulse inhibitory pathways directly via the central nervous system.

### SUMMARY OF THE INVENTION

The present invention provides an implantable cardioverter defibrillator system for detecting cardiac arrhythmias, delivering cardioversion shock therapy when indicated and preceding the cardioversion shock therapy with a prepulse inhibition (PPI) stimulus delivered directly to the spinal cord. The system for detecting arrhythmias, delivering cardioversion shock therapy, and delivering a PPI stimulus prior to shock therapy may be integrated into one implanted medical device with an associated system of one or more cardiac leads and at least one spinal cord stimulation (SCS) lead. Alternatively, the system may include two separate implantable devices, one for detecting arrhythmias and delivering shock therapy and a second for delivering a PPI stimulus upon receiving a command from the first device that a pain-inhibiting prepulse is needed.

In accordance with a method provided by the present invention, after detecting an arrhythmia, which may be an atrial or ventricular arrhythmia, the cardioverter defibrillator device selects an anti-arrhythmia therapy to be delivered according to selectable or programmable therapy options. If the therapy to be delivered is a cardioversion shock, a PPI stimulus trigger is generated. Output circuitry within the cardioverter defibrillator device may respond to the PPI stimulus trigger by generating a pulse of a predetermined or programmable energy. The PPI pulse is delivered directly to the spinal cord via the SCS lead. A timing control circuit controls the delivery of the PPI pulse at a given time interval prior to the delivery of the cardioversion shock. In an alternative embodiment, the PPI stimulus trigger signal is transmitted via a “body bus” to a separate PPI stimulation device implanted elsewhere in the patient's body. The PPI stimulation device receives the transmitted trigger signal and generates an output PPI pulse that is delivered directly to the spinal cord via a SCS lead.

By effectively inhibiting cardioversion shock pain through prepulse stimulation of the central nervous system, a patient is relieved of bearing the pain normally associated with cardioversion shocks. Cardioversion therapy may be more readily accepted by patients and physicians allowing broader application of the therapy for the treatment of arrhythmias.

### BRIEF DESCRIPTION OF THE DRAWINGS

FIG. 1A is a schematic illustration of an implantable cardioverter defibrillator and cardioversion pain inhibiting system implanted in a patient in accordance with one embodiment of the present invention.

FIG. 1B is a schematic illustration of an implantable cardioverter defibrillator and cardioversion pain inhibiting system implanted in a patient in accordance with an alternative embodiment of the present invention.

FIG. 2 is an illustration of an implantable cardioverter defibrillator (ICD) that may be included in the systems of FIGS. 1A and 1B and a partially cut-away view of a patient's heart depicting placement of an associated cardiac lead system.

FIG. 3A is a functional block diagram of the ICD of FIG. 1A.

FIG. 3B is a functional block diagram of the ICD and PPI stimulation device shown in FIG. 1B.

FIG. 4 is a flow diagram providing an overview of the operations included in a preferred embodiment of the present invention for delivering a PPI stimulus directly to the central nervous system prior to a cardioversion shock.

### DETAILED DESCRIPTION OF THE INVENTION

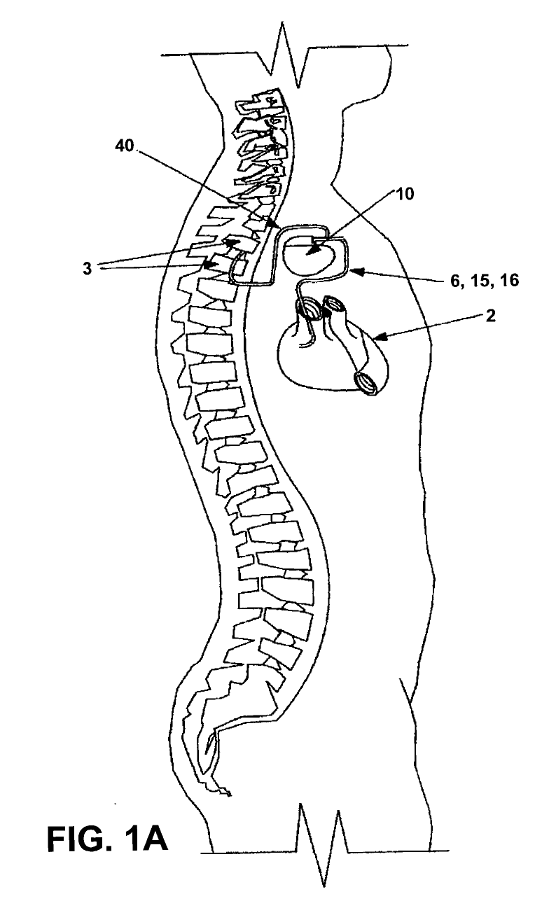

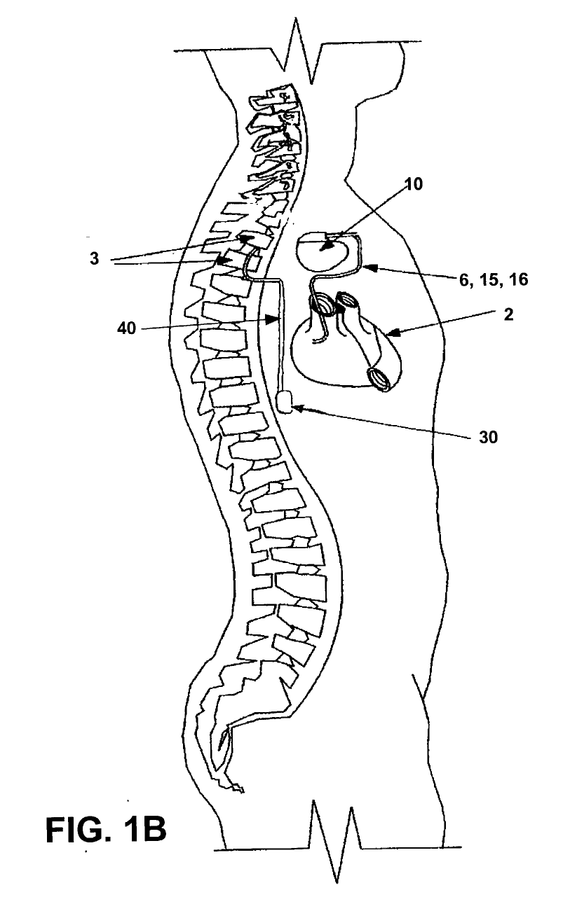

The present invention is aimed at providing a system and method for automatically delivering a prepulse inhibition (PPI) stimulus directly to the central nervous system to reduce or eliminate cardioversion shock pain. FIG. 1A is a schematic illustration of an implantable cardioverter defibrillator and cardioversion pain inhibiting system implanted in a patient in accordance with one embodiment of the present invention. The system includes a set of cardiac leads 6, 15, and 16 in communication with a patient's heart 2, and a spinal cord stimulation lead 40 in communication with the patient's spinal cord 3. The spinal cord stimulation (SCS) lead 40 may be provided as an epidural lead as generally described in commonly assigned U.S. Pat. No. 5,733,322 issued to Starkebaum and U.S. Pat. No. 6,308,103 issue to Gielen, both patents incorporated herein by reference in their entirety. Numerous types of spinal cord or epidural leads known for stimulating the spinal cord may be used successively with the present invention. Methods for implanting an epidural lead are generally disclosed in commonly assigned U.S. Pat. Nos. 5,255,691 and 5,360,441 issued to Otten, both patents incorporated herein by reference in their entirety. A SCS lead may include a plurality, e.g. four, spaced apart electrodes adapted to be placed in the epidural space adjacent to spinal segments. A PPI stimulus may be optimally effective in inhibiting cardioversion pain when delivered to the spinal cord generally in the region of the upper thoracic segments, such as spinal segments T1 and T2 as approximately depicted in FIG. 1A. The proximal end of SCS lead 40 is connected to an implantable cardioverter defibrillator device 10 that includes circuitry for delivering a PPI stimulus as will be described below.

FIG. 1B is a schematic illustration of an implantable cardioverter defibrillator and cardioversion pain inhibiting system implanted in a patient in accordance with an alternative embodiment of the present invention. Identically numbered components in FIG. 1B correspond to those in FIG. 1A, however, in the embodiment of FIG. 1B, circuitry for delivering a PPI stimulus is contained in a separate implantable device 30. PPI stimulation device 30 is controlled by commands transmitted to device 30 from ICD 10 through a “body bus,” as will be described in greater detail below. SCS lead 40 is connected to PPI stimulation device 30. An advantage of including PPI stimulation circuitry in a separate device is that device 30 may be implanted at a site different than ICD 10 which may allow SCS lead 40 to be more easily implanted and tunneled to PPI stimulation device 30. The length of SCS lead 40 may be reduced depending on the location of device 30.

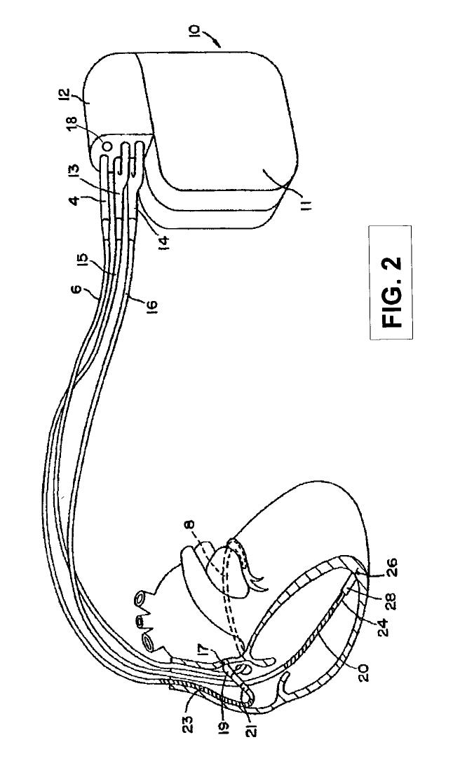

FIG. 2 is an illustration of an implantable cardioverter defibrillator (ICD) that may be included in the systems of FIGS. 1A and 1B and a partially cut-away view of a patient's heart depicting placement of an associated cardiac lead system. A connector block 12 receives the proximal end of a right ventricular lead 16, a right atrial lead 15 and a coronary sinus lead 6, used for positioning electrodes for sensing and stimulation in three or four heart chambers. Connector block 12 includes a port 18 for receiving SCS lead 40 for delivering a PPI stimulus directly to the spinal cord when PPI stimulus circuitry is included within ICD 10.

In FIG. 2, the right ventricular lead 16 is positioned such that its distal end is in the right ventricle for sensing right ventricular cardiac signals and delivering pacing or shocking pulses in the right ventricle. For these purposes, right ventricular lead 16 is equipped with a ring electrode 24, an extendable helix electrode 26 mounted retractably within an electrode head 28, and a coil electrode 20, each of which are connected to an insulated conductor within the body of lead 16. The proximal end of the insulated conductors are coupled to corresponding connectors carried by bifurcated connector 14 at the proximal end of lead 16 for providing electrical connection to the ICD 10.

The right atrial lead 15 is positioned such that its distal end is in the vicinity of the right atrium and the superior vena cava. Lead 15 is equipped with a ring electrode 21 and an extendable helix electrode 17, mounted retractably within electrode head 19, for sensing and pacing in the right atrium. Lead 15 is further equipped with a coil electrode 23 for delivering high-energy shock therapy. The ring electrode 21, the helix electrode 17 and the coil electrode 23 are each connected to an insulated conductor with the body of the right atrial lead 15. Each insulated conductor is coupled at its proximal end to a connector carried by bifurcated connector 13.

The coronary sinus lead 6 is advanced within the vasculature of the left side of the heart via the coronary sinus and great cardiac vein. The coronary sinus lead 6 is shown in the embodiment of FIG. 2 as having a defibrillation coil electrode 8 that may be used in combination with either the coil electrode 20 or the coil electrode 23 for delivering electrical shocks for cardioversion and defibrillation therapies. In other embodiments, coronary sinus lead 6 may also be equipped with a distal tip electrode and ring electrode for pacing and sensing functions in the left chambers of the heart. The coil electrode 8 is coupled to an insulated conductor within the body of lead 6, which provides connection to the proximal connector 4.

The electrodes 17 and 21 or 24 and 26 may be used for cardiac pacing as bipolar pairs, commonly referred to as a “tip-to-ring” configuration, or individually in a unipolar configuration with the device housing 11 serving as the indifferent electrode, commonly referred to as the “can” or “case” electrode. Housing 11 may also serve as a can electrode in combination with electrodes carried by SCS lead 40 for unipolar stimulation of the spinal cord. Housing 11 may also serve as a subcutaneous defibrillation electrode in combination with one or more of the defibrillation coil electrodes 8, 20 or 23 for defibrillation of the atria or ventricles. It is recognized that alternate lead systems may be substituted for the three cardiac lead system illustrated in FIG. 2.

Although three or four-chamber pacing, cardioversion and defibrillation capacity is not necessary for practicing the invention, a multi-chamber system is illustrated so as to indicate the scope of the invention. It is understood that the invention may normally be practiced with a single chamber atrial or ventricular cardioversion device, a dual chamber cardioversion device, or a multichamber cardioversion device. The device may include pacemaking capabilities in addition to arrhythmia detection and cardioversion therapy capabilities.

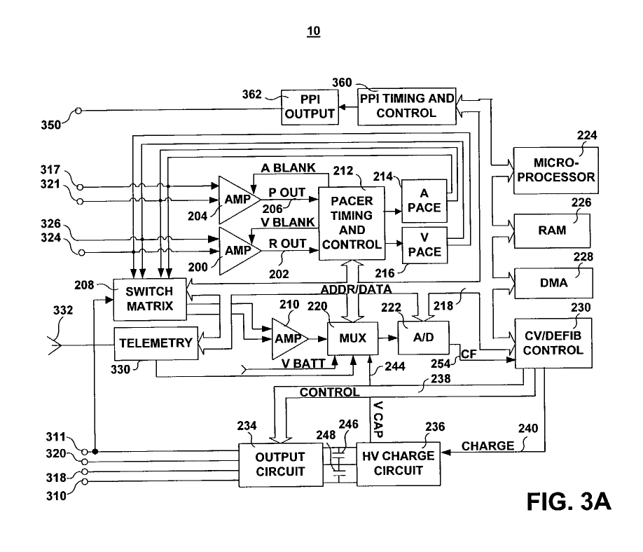

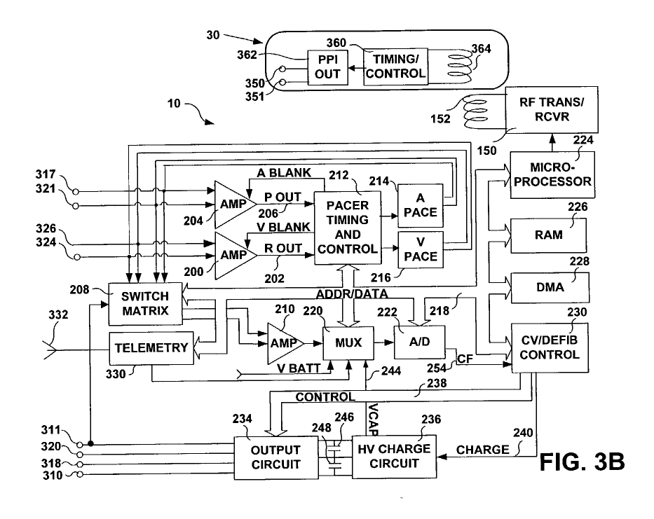

A functional block diagram of the ICD 10 of FIG. 1A is shown in FIG. 3A. This diagram should be taken as exemplary of the type of device with which the invention may be embodied and not as limiting. The disclosed embodiment shown in FIG. 3A is a microprocessor-controlled device, but the methods of the present invention may also be practiced with other types of devices such as those employing dedicated digital circuitry.

With regard to the electrode system illustrated in FIG. 2, the ICD 10 is provided with a number of connection terminals for achieving electrical connection to the cardiac leads 6, 15, and 16 and their respective electrodes. The connection terminal 311 provides electrical connection to the housing 11 for use as the indifferent electrode during unipolar stimulation or sensing. The connection terminals 320, 310, and 318 provide electrical connection to coil electrodes 20, 8 and 23 respectively. Each of these connection terminals 311, 320, 310, and 318 are coupled to the high voltage output circuit 234 to facilitate the delivery of high energy shocking pulses to the heart using one or more of the coil electrodes 8, 20, and 23 and optionally the housing 11.

The connection terminals 317 and 321 provide electrical connection to the helix electrode 17 and the ring electrode 21 positioned in the right atrium.

The connection terminals 317 and 321 are further coupled to an atrial sense amplifier 204 for sensing atrial signals such as P-waves. The connection terminals 326 and 324 provide electrical connection to the helix electrode 26 and the ring electrode 24 positioned in the right ventricle. The connection terminals 326 and 324 are further coupled to a ventricular sense amplifier 200 for sensing ventricular signals.

The atrial sense amplifier 204 and the ventricular sense amplifier 200 preferably take the form of automatic gain controlled amplifiers with adjustable sensing thresholds. The general operation of the ventricular sense amplifier 200 and the atrial sense amplifier 204 may correspond to that disclosed in U.S. Pat. No. 5,117,824, by Keimel, et al., incorporated herein by reference in its entirety. Whenever a signal received by atrial sense amplifier 204 exceeds an atrial sensing threshold, a signal is generated on the P-out signal line 206. Whenever a signal received by the ventricular sense amplifier 200 exceeds a ventricular sensing threshold, a signal is generated on the R-out signal line 202.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion and defibrillation functions of the ICD 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methodologies known in the art. A tachyarrhythmia recognition system is described in U.S. Pat. No. 5,545,186 issued to Olson et al., incorporated herein by reference in its entirety.

The telemetry circuit 330 receives downlink telemetry from and sends uplink telemetry to an external programmer, as is conventional in implantable anti-arrhythmia devices, by means of an antenna 332. Data to be uplinked to the programmer and control signals for the telemetry circuit 330 are provided by microprocessor 224 via address/data bus 218. In accordance with the present invention, control parameters for delivering a PPI stimulus may be downloaded to device 10 from an external programmer via telemetry circuit 330. PPI stimulus control parameters may include the pulse amplitude and width of the PPI stimulus and the time interval between a PPI stimulus and a succeeding cardioversion shock. Received telemetry is provided to microprocessor 224 via multiplexer 220. Numerous types of telemetry systems known for use in implantable devices may be used.

Circuitry illustrated in FIG. 3A includes an exemplary embodiment of circuitry dedicated to providing cardiac pacing, cardioversion and defibrillation therapies. The pacer timing and control circuitry 212 includes programmable digital counters which control the basic time intervals associated with various single, dual or multi-chamber pacing modes or anti-tachycardia pacing therapies delivered in the atria or ventricles. Pacer circuitry 212 also determines the amplitude of the cardiac pacing pulses under the control of microprocessor 224.

During pacing, escape interval counters within pacer timing and control circuitry 212 are reset upon sensing of R-waves or P-waves as indicated by signals on lines 202 and 206, respectively. In accordance with the selected mode of pacing, pacing pulses are generated by atrial pacer output circuit 214 and ventricular pacer output circuit 216. The pacer output circuits 214 and 216 are coupled to the desired electrodes for pacing via switch matrix 208. The escape interval counters are reset upon generation of pacing pulses, and thereby control the basic timing of cardiac pacing functions, including anti-tachycardia pacing.

The durations of the escape intervals are determined by microprocessor 224 via data/address bus 218. The value of the count present in the escape interval counters when reset by sensed R-waves or P-waves can be used to measure R-R intervals and P-P intervals for detecting the occurrence of a variety of arrhythmias.

The microprocessor 224 includes associated ROM in which stored programs controlling the operation of the microprocessor 224 reside. A portion of the random access memory 226 may be configured as a number of recirculating buffers capable of holding a series of measured intervals for analysis by the microprocessor 224 for predicting or diagnosing an arrhythmia. In response to the detection of tachycardia, anti-tachycardia pacing therapy can be delivered by loading a regimen from microcontroller 224 into the pacer timing and control circuitry 212 according to the type of tachycardia detected.

In the event that higher voltage cardioversion or defibrillation pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors 246 and 248 is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the pacer timing and control circuitry 212 by high voltage output circuit 234 via a control bus 238. The output circuit 234 determines the electrodes used for delivering the cardioversion or defibrillation pulse and the pulse wave shape.

In accordance with the present invention, prior to delivering the cardioversion pulse, a PPI stimulus is delivered under the control of PPI timing and control circuit 360. PPI timing and control circuit 360 is in communication with microprocessor 224 via data bus 218. When the voltage on VCAP line 244 reaches a predetermined value, which may be a value indicating the high voltage capacitors 246 and 248 are fully charged or, alternatively, are charged to a predetermined percentage of full charge, a PPI stimulus may be generated by PPI output circuit 362 under the control of timing and control circuit 360. PPI control circuit 360 determines the pulse width and pulse amplitude of the PPI stimulus, which may be programmable values received from telemetry circuit 330. The PPI stimulus generated by PPI output circuit 362 is delivered directly to the spinal cord via SCS lead 40 connected to a terminal 350 provided for electrically coupling SCS lead 40 to device 10.

In alternative embodiments, a dedicated PPI output circuit 362 may be eliminated, and a PPI stimulus may be generated by either of pacing output circuits 214 and 216 or high-voltage output circuit 234. Terminal 350 connected to SCS lead 40 may be selectively coupled to of output circuits 214, 216 or 234 by switch matrix 208 at the appropriate time for delivering a PPI stimulus. When either of pacing output circuits 214 or 216 is used for delivering the PPI stimulus, both the PPI stimulus pulse width and the pulse amplitude may be selected, under the control of PPI timing and control 360, from the settings available for atrial or ventricular pacing. When high-voltage output circuit 234 is used for delivering a PPI stimulus, the pulse amplitude will equal the amplitude of a high-voltage shock therapy, but the pulse width may be selected to be very narrow such that the PPI stimulus is weaker than the succeeding high-voltage shock. The use of high-voltage defibrillation circuitry for delivery of an atrial or ventricular prepulse is generally described in the previously incorporated '418 patent.

FIG. 3B is a functional block diagram of the ICD 10 and PPI stimulation device 30 shown in FIG. 1B. In FIG. 3B, identically numbered components correspond to those in FIG. 3A, however in FIG. 3B, PPI timing and control circuit 360 and PPI output circuit 362 for delivering a PPI stimulus are removed from ICD 10 and included in separate PPI stimulation device 30. Device 30 preferably receives commands from ICD 10 via a “body bus,” as generally disclosed in U.S. Pat. No. 4,987,897, issued to Funke, incorporated herein by reference in its entirety. ICD 10 is provided with a transmitter 150 and transducer 152 for transmitting frequency modulated signals from ICD 10 to PPI stimulation device 30. Modulated signals for transmission from ICD 10 to device 30 include information relating to PPI stimulus pulse amplitude and width, which information is provided to transmitter 150 from microprocessor 224. Transmitted signals are received by transducer 364 of device 30 and demodulated by timing and control circuit 360. Device 30 may optionally include a transmitter for transmitting signals back to ICD 10. Device 30 receives a PPI stimulus trigger command from ICD 10 at the appropriate time for delivering a PPI stimulus, prior to a cardioversion shock. The PPI stimulus is delivered by PPI output circuit 362 with a pulse width and amplitude set by timing and control circuit 360 based on commands received from ICD 10. The PPI stimulus is delivered directly to the central nervous system via terminal 350 connected to SCS lead 40 and terminal 351, which may be connected to the housing of device 30 for serving as a can electrode during unipolar PPI stimulation. Alternatively, terminal 351 may be provided for connection to one or more anode electrodes included on SCS lead 40 for bipolar stimulation of the spinal cord.

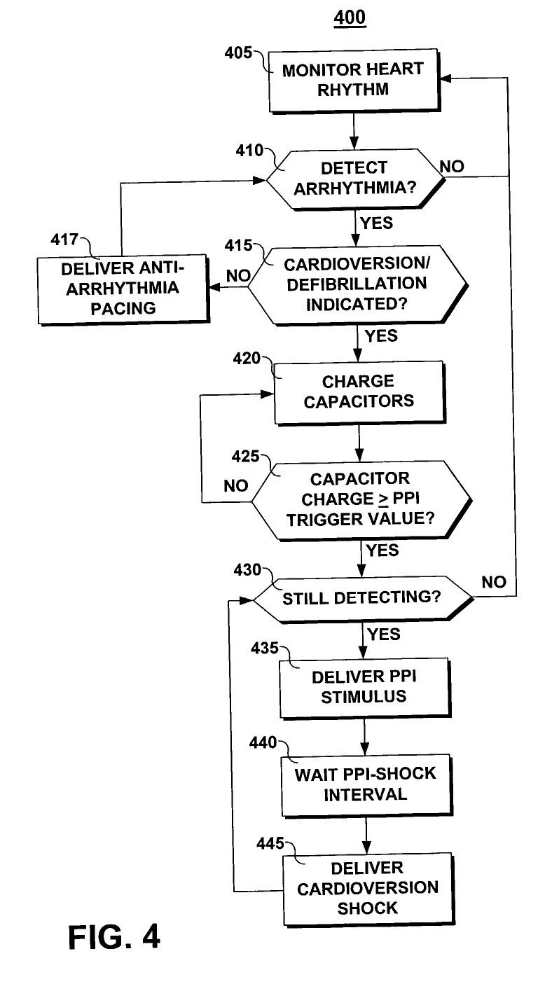

In FIG. 4 a flow diagram is shown providing an overview of the operations included in a preferred embodiment of the present invention for delivering a PPI stimulus directly to the central nervous system prior to a cardioversion shock. At step 405, cardiac signals are sensed to determine various intervals associated with P-waves and R-waves by pacer timing and control 212. Step 405 is executed continuously to monitor the heart's rhythm at all times, except for during blanking intervals applied to ventricular and atrial sense amplifiers 200 and 204 during pacing or shocking pulse delivery. If an arrhythmia is detected at decision step 410, an appropriate anti-arrhythmia therapy is selected. Depending on the type of arrhythmia detected, a cardioversion shock therapy may not be indicated. For example, when a tachycardia detection is made, programmed therapies may include tiered therapies beginning with anti-tachycardia pacing therapies which are attempted prior to delivering cardioversion shocks. If a cardioversion or defibrillation shock therapy is not indicated at decision step 415, the appropriate anti-arrhythmia pacing therapy is delivered at step 417. If the arrhythmia is terminated (as determined at step 410), the method 400 returns to step 405 and continues monitoring the heart rhythm.

If a cardioversion or defibrillation shock therapy is indicated in response to a detected arrhythmia, as determined at decision step 415, charging of the high voltage capacitors is initiated at step 420. After the capacitor charge has reached a predetermined PPI stimulus trigger value, as determined at step 425, microprocessor 224 verifies that an arrhythmia is still being detected at decision step 430, and then triggers the delivery of the PPI stimulus at step 435. A PPI stimulus trigger is preferably generated upon full charging of the high-voltage capacitors such that the capacitors are ready to deliver a cardioversion shock after a short time delay, e.g. after less than 500 ms, after a PPI stimulus is delivered. Alternatively, the PPI stimulus trigger may be generated once high-voltage capacitors are charged to a certain percentage of full charge, for example 90% fully charged, so that by the time the PPI stimulus has been delivered and the PPI-shock delay period has elapsed, the capacitors are fully charged and the cardioversion shock may be immediately delivered. In this alternative embodiment, the PPI stimulus would be generated by either dedicated or pacing output circuitry, not the high-voltage output circuitry since high-voltage capacitors would still be charging during PPI stimulus delivery. As described above, a PPI stimulus may be delivered from output circuitry included in device 10 according to the embodiment of FIG. 3A. Alternatively, the PPI stimulus trigger may generate a telemetry signal transmitted by ICD 10 to PPI stimulation device 30 which in turn triggers a PPI stimulus to be delivered from PPI stimulation device 30 according to the embodiment of FIG. 3B. The amplitude, duration, and wave shape of the PPI stimulus may be set according to fixed or programmable values and may be selected based on an individual patient's response. Generally monophasic or biphasic pulses or pulse trains could be utilized for a PPI stimulus. If the arrhythmia has self-terminated during capacitor charging, as determined at decision step 430, the method 400 returns to step 405 and continues monitoring the heart rhythm.

If arrhythmia detection is still occurring at step 430, the PPI stimulus is delivered at step 435. Timer and control circuitry 212 then sets a PPI-shock delay interval that must expire prior to delivering the cardioversion shock at step 445. The time interval between the PPI stimulus and the cardioversion shock may be fixed or programmable according to an individual patient's response. The time interval required for an optimal PPI effect may vary between approximately 20 and 500 ms, and is typically on the order of approximately 100 ms.

After delivering the cardioversion shock at step 445, method 400 returns to step 430 to determine if an arrhythmia is still detected. If so, steps 435 through 445 are repeated. If the arrhythmia is successfully terminated, method 400 returns to step 405 to continue monitoring the heart rhythm.

Thus a system and method for delivering a prepulse inhibition stimulus directly to the central nervous system prior to a cardioversion shock therapy has been disclosed. The embodiments described herein are considered the preferred embodiments contemplated to date and are intended to be exemplary, not limiting, with regard to the following claims.

What is claimed is:

1. A system for delivering a prepulse inhibition stimulus implemented with a medical device comprising:
means for detecting arrhythmia;
means for confirming arrhythmia needing cardioversion shock;
means for delivering a prepulse inhibition stimulus; and
means for delivering a cardioversion shock;
said means for detecting, means for confirming and means for delivering being in cooperative communication to deliver the prepulse inhibition stimulus in temporally spaced interval prior to delivering a cardioversion shock.

2. An implantable medical device having a plurality of electrodes to stimulate cardiac and central nervous tissue to inhibit pain perception comprising:
a cardioversion defibrillation device;
a plurality of leads in operable electrical connection with said device; and
means for coordinating delivery of a prepulse inhibition in temporally spaced interval prior to delivery of a cardioversion shock via one of said plurality of leads.
